(19) United States
(12) Patent Application Publication
(10) Pub. No.: US 2007/0179331 A1
(43) Pub. Date: Aug. 2, 2007

(54) Method for hydrocarbon isomerization

(76) Inventors: David E. W. Vaughn, State College, PA (US); Karl G. Strohmaier, Port Murray, NJ (US); Wayne R. Kliewer, Clinton, NJ (US)

Correspondence Address: ExxonMobil Research & Engineering Company, P.O. Box 900, 1545 Route 22 East, Annandale, NJ, 08801-0900, US

(21) Appl. No.: 10/550,485

(22) PCT Filed: Apr. 2, 2004
(86) PCT No.: PCT/US04/10220

§ 371 Date: Jul. 21, 2006

Related U.S. Application Data
(60) Provisional application No. 60/462,020, filed on Apr. 11, 2003.
(60) Provisional application No. 60/462,019, filed on Apr. 11, 2003.

(51) Int. Cl. C07C 5/13

(52) U.S. Cl. 585/750

(57) ABSTRACT

An improved isomerization process for hydrocarbon feedstreams through the use of aqueous-treated ferrierite catalysts is disclosed.

### FIELD OF THE INVENTION

The present invention is directed at an improved hydrocarbon isomerization process. More particularly, the present invention is directed at an improved isomerization process for hydrocarbon feedstrearns through the use of aqueous-treated ferrierite catalysts.

### BACKGROUND OF THE INVENTION

The use of steamed or water treated catalysts in isomerization processes is described in the art and literature. U.S. Pat. No. 4,418,235 discloses the use of zeolites with a pore dimension greater than about 5 Angstroms, preferably 10-membered rings, with a silica to alumina ratio of at least 12 and a constraint index of about 1 to about 12. These zeolites undergo a treatment with steam or water prior to use and are used in an acid catalyzed conversion process.

U.S. Pat. No. 4,374,296 discloses the use of zeolites with a pore dimension greater than about 5 Angstroms, preferably 10-membered rings, with a silica to alumina ratio of greater than 12 and a constraint index of about 1 to about 12. The catalysts undergo a controlled treatment to enhance the acidity, expressed as alpha, to about 300. These are used in the hydroisomerization of a C₄ to C₈ paraffin.

Other methods, which emulate the methods of U.S. Pat. No. 3,293,192, have focused on severe treatments that target nation of the zeolite framework. While still other methods such as viewed by Kerr, American Chemical Society Advanced Chemical Series, vol. 121, 219 (1973) have targeted extraction of framework aluminum the use of chemical extraction.

All of the above referenced patents are hereby incorporated by reference.

However, there still exists a need in the art for an improved process for isomerizing a hydrocarbon feedstream.

### SUMMARY OF THE INVENTION

The present invention is directed at a process to isomerize hydrocarbon feedstreams comprising:
a) contacting a hydrocarbon feedstream with an aqueous-treated catalyst comprising ferrierite, or a zeolite isostructural to ferrierite, under hydroisomerization conditions including:
   i) temperatures of about 400 to about 800° F. (205° C. to about 430° C.); and
   ii) pressures of about 400 to about 2000 psig (2860 to about 13890 kPa); wherein said aqueous-treated catalyst is treated under conditions such that the aqueous-treated catalysts show removal of sorbed ammonia at a temperature about 248° F. (120° C.) lower than the same untreated catalyst.

In one embodiment the hydrocarbon feedstream is C₁₀₊ hydrocarbon feedstream.

In another embodiment the hydrocarbon feedstream is C₉₋ hydrocarbon feedstream.

### DETAILED DESCRIPTION OF THE PRESENT INVENTION

Ferrierite is a 10-ring mineral zeolite that is readily synthesized. Ferrierite is useful in many hydrocarbon conversion reactions such as cracking of low octane paraffins or selectively sorbing them. Ferrierite is also useful for converting linear paraffins to olefins and for the separation of single from multi-branched olefins and paraffins. Thus, it would be beneficial to improve the selectivity of ferrierite.

The present invention utilizes aqueous-treated catalysts comprising ferrierite, or a zeolite isostructural to ferrierite such as, for example, FU-9, ISI-6, NU-23 and ZSM-35, in the hydroisomerization of hydrocarbon feedstreams. The hydrocarbon feedstream is contacted with the aqueous-treated catalyst under hydroisomerization conditions that include temperatures from about 400 to about 800° F. (205° C. to about 430° C.), and pressures from about 400 to about 2000 psig (2860 to about 13890 kPa). Aqueous-treated, as used herein, is meant to refer to a catalyst that has been subjected to a treatment with an aqueous solution prior to use, and untreated is meant to refer to a catalyst that has not been subjected to an aqueous treatment.

Feedstreams suitable for use in the present process are any hydrocarbon streams, however, C₁₀₊ hydrocarbon feedstreams, and C₉₋ are those which are commonly used. C₁₀₊ hydrocarbon feedstreams typically boil in the range of about 345 to about 1050° F. (173 to about 565° C.), preferably about 650 to about 1000° F. (343 to about 538° C.), and more preferably about 750 to about 950° F. (400 to about 510° C.). Feedstreams boiling in the C₉₋ typically boil below about 305° F. (155° C.), preferably those boiling within the C₄ to C₉ range are used. As used herein, these streams include those boiling in the range of about 0 to about 150° F. (32 to about 305° C.). By using the aqueous-treated catalysts disclosed herein, C₁₀₊ hydrocarbon feedstreams have shown an improvement in cold flow properties, and C₉₋ hydrocarbon feedstreams have shown an increase in octane.

Ferrierite is generally considered a molecular sieve having the characteristics of a unidimensional 10 ring zeolite, i.e. a medium pore zeolite having unidimensional channels comprising 10 member rings. Zeolites are porous crystalline materials and medium pore zeolites are generally defined as those having a pore size of about 5 to about 7 Angstroms, such that the zeolite freely sorbs molecules such as n-hexane, 3-methylpentane, benzene and p-xylene. Another common classification used for medium pore zeolites involves the Constraint Index test which is described in U.S. Pat. No. 4,016,218, which is hereby incorporated by reference. Medium pore zeolites typically have a Constraint Index of about 1 to about 12, based on the zeolite alone without modifiers and prior to treatment to adjust the diffusivity of the catalyst.

Ferrierite can be readily synthesized, and the ferrierite catalysts used herein can be synthesized with or without a template. The templates used to synthesize ferrierite are typically organic in nature. Non-limiting examples of templates include tetramethylammonium, ethylenediamine, pyrrolidines, piperidines, etc. It is preferred that the ferrierite catalysts used herein be synthesized using an organic template.

It is also preferred that the ferrierite catalysts used herein contain at least one Group VIII metal, preferably a Group VIII noble metal, more preferably Pt and Pd, and most preferably Pt. The metals are present in an amount from about 0.05 to about 2.0 wt. %, preferably from about 0.1 to about 1.0 wt. %, based on the total weight of the catalyst. The metals can be incorporated through the use of any suitable means or technique known, such as, for example, incipient wetness.

The present method involves an aqueous treatment wherein the ferrierite catalysts described above are submerged in an aqueous solution to form a slurry. The aqueous solution can be about 100% water or the aqueous solution can comprise water and a gas or other material that is substantially inert to the ferrierite catalysts. It is preferred that the aqueous solution be about 100% water, more preferably deionized water.

After the catalyst has been submerged in the aqueous solution, the pH of the slurry is adjusted. The pH can be adjusted through the use of any suitable conventional method or process. However, the pH is typically adjusted, or maintained, by the addition of a material that does not have a deleterious effect on the catalyst or the catalysts' functionality after the aqueous treatment. Preferably, an effective amount of an acid such as hydrochloric acid, preferably a dilute acid, is added to lower the pH of the slurry or an effective amount of a basic solution such as dilute aqueous ammonium hydroxide is added to raise the pH of the slurry. The pH of the slurry is adjusted to a desired pH in the range of about 2 to about 7, preferably to about 3 to about 5. Thus, by an effective amount of an acid or basic solution, it is meant that amount of acid or basic solution needed to adjust the pH of the aqueous solution to the desired pH.

After the pH has been adjusted to the desired pH, the slurry is heated to a predetermined temperature, ranging from about 210° F. to about 575° F. (100 to about 300° C.), preferably from about 284° F. to about 500° F. (140 to about 260° C.), more preferably from about 355° F. to about 428° F. (180 to about 220° C.). The catalyst is subjected to the aqueous treatment conditions for an effective amount of time, which is typically less than about 24 hours, preferably less than about 20 hours, and more preferably about 12 to about 18 hours. As previously stated, the present method does not target dealumination, and the ferrierite catalysts, after being effectively treated, do not show any evidence of dealumination.

By “effectively treated” it is meant that the resulting aqueous-treated catalyst is capable of desorbing sorbed ammonia at temperatures lower than the same untreated catalyst. Typically the aqueous-treated catalyst is capable of desorbing sorbed ammonia at temperatures about 248° F. (120° C.) lower than the untreated catalyst, preferably from about 76° F. to about 248° F. (80 to about 120° C.) lower than the untreated catalysts, more preferably about 194° F. to about 230° F. (90 to about 110° C.), and most preferably about 203° F. to about 221° F. (95 to about 105° C.). The decrease in temperature at which sorbed ammonia is desorbed is accompanied by a reduction of catalytic acidity. Thus, the aqueous-treated catalyst has less of a tendency towards non-selective cracking and shows improved isomerization characteristics. Therefore, an effectively treated catalyst is one that demonstrates desorption of sorbed ammonia at temperatures lower than an untreated catalyst, a decrease in tendency towards non-selective cracking, and improved isomerization characteristics. The reason for the improved desorption properties resulting from the aqueous treatment is unknown. However, the inventors hereof, while not wishing to be limited by theory, believe that changes in surface properties, structural annealing to eliminate structural defects or changes in metal dispersion, or combinations of these account for the aqueous-treated catalyst having a decreased tendency towards non-selective cracking, reduction of the temperature at which sorbed ammonia is removed, and improved isomerization characteristics.

Also, as previously stated, it is preferred that the catalysts used herein contain at least one Group VIII metal, preferably a Group VIII noble metal, and most preferably Pt. The at least one Group VIII metal can be added to the ferrierite catalysts before or after they have been subjected to the presently disclosed method. However, it is preferred that the ferrierite catalysts be subjected to the presently disclosed method subsequent to the incorporation of the at least one Group VIII metal.

The ferrierite catalysts can also be combined with a suitable binder or matrix material. Such materials include active and inactive materials such as clays, silica, and/or metal oxides such as alumina. Non-limiting examples of naturally occurring clays that can be composited include clays from the montmorillonite and kaolin families including the subbentonites, and the kaolins commonly known as Dixie, McNamee, Georgia, and Florida clays. Others in which the main mineral constituent is halloysite, kaolinite, dickite, nacrite, or anauxite may also be used. The clays can be used in the raw state as originally mixed or subjected to calcination, acid treatment, or chemical modification prior to being combined with the ferrierite.

Additionally, the ferrierite catalyst can also comprise a porous matrix or binder material such as silica-alumina, silica-magnesia, silica-zirconia, silica-thoria, silica-beryllia, or silica-titania. The ferrierite can also comprise a ternary composition such as silica-alumina-thoria, silica-alumina-zirconia, silica-alumina-magnesia, and silica-magnesia-zirconia.

It is preferred that the porous matrix or binder material comprises silica, alumina, or a kaolin clay. It is more preferred that the binder material comprise alumina. In this embodiment the alumina is present in a ratio of less than about 15 parts ferrierite to one part binder, preferably less than about 10, more preferably less than about 5, and most preferably about 2.

In general, the present invention is practiced by contacting a hydrocarbon feedstream as described above with a aqueous-treated ferrierite catalyst, as described above, under hydroisomerization conditions. The hydroisomerization conditions include temperatures 400 to about 800° F. (205° C. to about 430° C.), and pressures from about 400 to about 2000 psig (2860 to about 13890 kPa), hydrogen circulation rates between about 1000 and 5000 scf/bbl (178.1 to about 890.5 m³/m³), and space velocities between about 0.25 and 2.0.

The use of the aqueous-treated catalysts improves the product selectivity of the hydroisomerization process by more than about 20 percent, preferably more than about 30 percent, more preferably about 50 percent, and most preferably more than about 50%.

The above description is directed to one embodiment of the present invention. Those skilled in the art will recognize that other embodiments that are equally effective could be devised for carrying out the spirit of this invention.

The following examples will illustrate the effectiveness of the present process, but are not meant to limit the present invention.

### EXAMPLES

### Example 1 (Compararive)

A base untreated ferrierite catalyst, Zeolyst (CBV-914B), having a Si/Al ratio of about 10 was obtained commercially. This catalyst was presumably made using an organic template, outlined in U.S. Pat. Nos. 4,252,499 and 4,942,027. The untreated ferrierite was prepared by calcining to remove the template, ammonium exchanged, and exchanged with a solution of Pt(NH₃)4Cl₂4H₂O to yield a catalyst having 0.5 wt. % Pt, based on the total weight of the catalyst. The untreated ferrierite exchanged catalyst was then calcined in air at 115° C., followed by a programmed calcination comprising heating for 30 minutes at 115° C., increasing the temperature at 0.5° C./minute to 450° C., then holding the temperature at 450° C. for 2 hours. The catalyst was then cooled, pilled, ground, and sieved into particles ranging from about 0.85 to about 2.0 mm for catalyst testing. The catalyst was dried for at least 30 minutes in a 250° C. oven. A 0.50 g portion of the dried ferrierite was then mixed with 5.0 g of about 0.25-0.66 mm quartz chips to form a catalyst charge. The catalyst charge was loaded into a 1 cm diameter, stainless steel, downflow, automated reactor equipped with an on-line gas chromatogram containing a 50 m capillary column. The ferrierite samples used herein were then pretreated by ramping the temperature to 150° C. and holding that temperature for 30 minutes under a flow of 200 SCCM dry nitrogen. The nitrogen was switched to hydrogen and the temperature ramped to 350° C. and held there for 60 minutes to reduce the platinum. The temperature was reduced to 240° C. and hydrogen and decane were introduced at a rate of 29 WHSV (weight hourly space velocity), while a total pressure of 200 psig and a H₂:feed ratio of 10 were maintained. The temperature was increased in 20° C. increments to a maximum of 400° C., and held at each increment for a period of 30 minutes. The conversion of decane was measured at each temperature, and the results are given in Table 1 below.

At a standard comparative temperature of 320° C., this untreated ferrierite catalyst converted 69% of the feed, wherein 24% were decane isomers and 41% were cracked products. In evaluating the temperature dependence of activity and selectivity, this was the maximum isomerization yield observed with this catalyst.

### Example 2

A 5 g sample of the untreated ferrierite catalyst was slurried in 50 g of deionized water in a 125 Teflon autoclave liner (Parr 4748 acid digestion bomb). The pH was adjusted to 2 by adding dilute HC1, and the liner was capped and placed in the bomb container and sealed. The bomb was rotated at 12 rpm in the presence of air in an air oven at 200° C. and held at that temperature for about 18 hours. The bomb was then cooled, and the contents removed. The contents of the bomb were filtered to retrieve the ferrierite catalyst particles and the catalyst particles were washed with deionized water and dried at 115° C. The ferrierite catalyst was then prepared for testing and evaluated using the same catalytic procedure and preparation methods described in Example 1. The conversion of decane was measured, and the results are given in Table 1 below.

At a standard comparative temperature of 320° C., this treated ferrierite catalyst converted 23% of the feed, wherein 17% ere decane isomers and 6% were cracked products. In this case, maximum isomerization was observed at 380° C. with a conversion of 66% of the feed, wherein 39% were decane isomers and 27% cracked products. The activity of this catalyst has been moderated, but its selectivity to desired products has been enhanced compared to the base catalyst of Example 1.

### Example 3

A 5g sample of the base ferrierite catalyst from Example 1 was treated the same as the catalyst of Example 2, except that the pH was adjusted to 7 with a few drops of dilute aqueous ammonium hydroxide. The treated catalyst was subjected to the standard catalyst preparation and treatment procedures discussed in Examples 1 and 2.

At a standard comparative temperature of 320° C., this treated ferrierite catalyst converted 45% of the feed, wherein 29% were decane isomers and 16% were cracked products. In this case, maximum isomerization was observed at 340° C. with a conversion of 61% of the feed, wherein 34% were decane isomers and 27% cracked products.

### Examples 4-11

Samples of ferrierite catalysts were prepared according to the procedure outlined in Examples 1, 2, and 3. However, the treatment temperature and pH was varied, as shown in Table 1 below.

TABLE 1

| | | 320° C. Data | | | Maximum Isomerization Selectivity Data | | | |
|---|---|---|---|---|---|---|---|---|
| | | | | % | | | | % |
| | Treatment | % | % Decane | Cracked | Temperature | % Decane | % | Cracked |
| Example # | T° C./pH | Conversion | Isomers | Products | (° C.) | Isomers | Conversion | Products |
| 1 | NONE | 69 | 24 | 41 | 320 | 27 | 69 | 41 |
| 2 | 200/2 | 23 | 17 | 6 | 380 | 39 | 66 | 27 |
| 3 | 200/7 | 45 | 29 | 16 | 340 | 34 | 61 | 27 |
| 4 | 165/4 | 50 | 32 | 18 | 340 | 37 | 66 | 30 |
| 5 | 165/5 | 48 | 37 | 11 | 340 | 45 | 65 | 20 |
| 6 | 165/2 | 38 | 29 | 9 | 360 | 42 | 67 | 24 |
| 8 | 240/4 | 21 | 18 | 3 | 380 | 45 | 68 | 20 |
| 9 | 200/3.4 | 34 | 25 | 9 | 360 | 42 | 70 | 25 |
| 10 | 200/3.6 | 32 | 26 | 6 | 360 | 45 | 60 | 16 |
| 11 | 200/5 | 50 | 40 | 10 | 360 | 48 | 76 | 27 |
| 12 | 200/3 | 33 | 26 | 8 | 360 | 42 | 62 | 20 |

It is clear from the data contained in Table 1 that the controlled aqueous treatments between a pH of about 2 to about 7 in the temperature range of about 140 to about 260° C. provides favorable activity and selectivity improvements over the prior art ferrierite catalysts, represented here by the commercial catalyst evaluated in Example 1. As can be seen from the data contained in Table 1, optimized performance of the water-treated ferrierite catalysts is achieved in the preferred pH range of between about 3 and 5, and over the more preferred temperature range of between about 180 to about 220° C.

1. A process to isomerize hydrocarbon feedstreams comprising:
a) contacting a hydrocarbon feedstream with a aqueous-treated catalyst comprising ferrierite, or a zeolite isostructural to ferrierite, under hydroisomerization conditions including:
i) temperatures of about 400 to about 800° F. (205° C. to about 430° C.); and
ii) pressures of about 400 to about 2000 psig (2860 to about 13890 kPa);
wherein after the above-described method, said catalyst desorbs sorbed ammonia at a temperature about 248° F. (120° C.) lower than the same untreated catalyst before the above-described method.

2. The process according to claim 1 wherein said hydrocarbon feedstream is a C₁₀₊ hydrocarbon feedstream boiling in the range of about 345° F. to about 1050° F.

3. The process according to claim 1 wherein said hydrocarbon feedstream is a C₉₋ hydrocarbon feedstream boiling below about 345° F.

4. The process according to any of the claim 1 wherein said aqueous-treated catalyst further comprises about 0.05 to about 2.0 wt. %, based on the catalyst, of at least one Group VIII metal.

5. The process according to claim 4 wherein said Group VIII metal is a Group VIII noble metal.

6. The process according to claim wherein said Group VIII metal is Pt.

7. The process according to claim 6 wherein said aqueous-treated catalyst is subjected to a water treatment comprising submersing said aqueous-treated in catalyst in water for less than about 24 hours at a temperature of about 21 0° F. to about 575° F. (100 to about 300° C.).

8. The process according to claim 7 wherein the pH of said water is adjusted to about 2 to about 7 through the addition of an acidic or basic material that does not have a deleterious effect on said aqueous-treated catalyst.

9. The process according to claim 8 wherein said basic material is dilute aqueous ammonium hydroxide, and said acidic material is dilute hydrochloric acid.

10. The process according to claim 8 wherein the product selectivity of the hydroisomerization process improves by more than about 20%.

11. The process according to claim 9 wherein the product selectivity of the hydroisomerization process improves by more than about 30%.

12. The process according to claim 10 wherein the product selectivity of the hydroisomerization process improves by more than about 50%.

13. The process according to any of the claim 6 wherein said aqueous-treated catalyst is treated after the addition of the metals.

14. The process according to claim 13 wherein said aqueous-treated catalyst further comprises at least one binder or matrix material selected from clays, silica, and alumina.

15. The process according to claim 14 wherein said binder or matrix material is alumina present in a ratio of less than about 15 parts zeolite to one part binder.

16. The process according to claim 9 wherein said water treatment does not result in the dealumination of said ferrierite.

17. A process to isomerize hydrocarbon feedstreams comprising:
a) contacting a hydrocarbon feedstream with a aqueous-treated catalyst comprising ferrierite, or a zeolite isostructural to ferrierite, and about 0.05 to about 2.0 wt. % of at least one Group VIII metal, based on the weight of the catalyst, under hydroisomerization conditions including:
i) temperatures of about 400 to about 800° F. (205° C. to about 430° C.); and
ii) pressures of about 400 to about 2000 psig (2860 to about 13890 kPa);
wherein said aqueous-treated catalyst is treated under conditions such that the aqueous-treated catalysts show removal of sorbed ammonia at a temperature about 194° F. to about 230° F. (90 to about 110° C.) lower than the same untreated catalyst.

18. The process according to claim 17 wherein said hydrocarbon feedstream is a C₁₀₊ hydrocarbon feedstream boiling in the range of about 345° F. to about 1050° F.

19. The process according to claim 17 wherein said hydrocarbon feedstream is a C₉₋ hydrocarbon feedstream boiling below about 345° F.

20. The process according to claim 17 wherein said Group VIII metal is a Group VIII noble metal.

21. The process according to claim 20 wherein said Group VIII metal is Pt.

22. The process according to claim 21 wherein said aqueous-treated catalyst is subjected to an aqueous treatment comprising submersing said aqueous-treated in catalyst in water for less than about 20 hours at a temperature of 284° F. to about 500° F. (140 to about 260° C.).

23. The process according to claim 22 wherein the pH of said water is adjusted to about 3 to about 5 through the addition of dilute aqueous ammonium hydroxide or dilute hydrochloric acid.

24. The process according to any of the claim 23 wherein said aqueous-treated catalyst is treated after the addition of the at least one Group VIII metal.

25. The process according to claim 21 wherein said aqueous-treated catalyst further comprises at least one binder or matrix material selected from clays, silica, and alumina.

26. The process according to claim 24 wherein said aqueous treatment does not result in the dealumination of said ferrierite.

27. The process according to claim 26 wherein the product selectivity of the hydroisomerization process improves by more than about 20%.
